(19) United States
(12) Patent Application Publication
(10) Pub. No.: US 2002/0049544 A1
(43) Pub. Date: Apr. 25, 2002

(54) Image analysis for phenotyping sets of mutant cells

(75) Inventors: Corey E. Nislow, San Francisco, CA (US); Nolan H. Sigal, Los Altos, CA (US); David G. Drubin, Berkeley, CA (US); Cynthia L. Adams, Berkeley, CA (US)

Correspondence Address: BEYER WEAVER & THOMAS LLP, P.O. BOX 778, BERKELEY, CA, 94704-0778, US

(73) Assignee: Cytokinetics, Inc.

(21) Appl. No.: 09/888,063

(22) Filed: Jun. 22, 2001

Related U.S. Application Data
(60) Provisional application No. 60/213,850, filed on Jun. 23, 2000.

(51) Int. Cl. G01N 33/48

(52) U.S. Cl. 702/19

(57) ABSTRACT

A method described herein phenotypes a set of mutant strains in a quantitative manner. Specifically, the method characterizes a cellular and subcellular architecture of mutant alleles grown in a variety of conditions using various morphological and molecular markers, combined with automated image acquisition and analysis. Phenotypic features may include the cytoskeleton, organelles, cell morphology, DNA replication state, the relationship of these features to each other, etc. From these features a quantitative “fingerprint” can be generated for each phenotype. This quantitative phenotypic information is made available in a database that links genotype to phenotype. Genes characterized in this manner may be clustered into functional categories, pathways, higher order protein assemblies, and the like.

### CROSS-REFERENCE TO RELATED APPLICATIONS

This application claims priority under 35 USC § 119(e) from U.S. Provisional Patent application No. 60/213,850, filed Jun. 23, 2000, and titled “IMAGE ANALYSIS FOR PHENOTYPING SETS OF MUTANT CELLS.” The content of that Provisional Patent Application is incorporated herein by reference for all purposes.

### BACKGROUND OF THE INVENTION

The present invention pertains to systems and methods for obtaining, analyzing and using images of specific cells. More specifically, the present invention pertains to systematically characterizing phenotypes of deletion mutants congenic to a single parent.

Genes of various organisms are being identified at an ever-increasing rate. Frequently a gene's structure is identified long before its function is accurately characterized. Many such genes may be important in disease states. One daunting task of the human genome project is to connect the various genes being discovered with particular diseases. Ultimately, such information can be applied to develop new drugs for treating the particular diseases.

Somewhat surprisingly, between 40 and 45 percent of yeast genes have homologs in humans. The entire yeast genome has now been mapped and sequenced. Common Baker's yeast, Saccharomyces cerevisiae, has been analyzed and systematically modified by the Saccharomyces cerevisiae Deletion Consortium to yield a complete set of congenic deletion mutants. In the complete set of deletion mutants, a single gene has been completely deleted in each mutant strain. Saccharomyces cerevisiae has approximately 6200 genes. Of these, approximately 17 percent are essential. In other words, if any such gene is deleted, the organism will be inviable. For the remaining genes, approximately one-third are of unknown function. One way to assign function and gain valuable biological knowledge is to carefully phenotype each deletion mutant.

Accordingly, it would be desirable to characterize the various strains from the Consortium (or another set of deletion strains) based on phenotype to ascertain function.

### SUMMARY OF THE INVENTION

This invention offers a method of phenotyping a set of mutant strains in a quantitative manner. Specifically, the invention characterizes a cellular and subcellular architecture of deletion alleles grown in a variety of conditions using various morphological and molecular markers, combined with automated image acquisition and analysis. Phenotypic features may include the cytoskeleton, organelles, cell morphology, DNA replication state, the relationship of these features to each other, etc. From these features a quantitative “fingerprint” can be generated for each phenotype. This quantitative phenotypic information is made available in a database that links genotype to phenotype. Genes characterized according to this invention may be clustered into functional categories, pathways, higher order protein assemblies, and the like.

One aspect of the invention provides a method of analyzing a collection of genetically modified cell strains that are congenic with a single parent strain. This method may be characterized by the following sequence: (a) receiving images of phenotypes for each of the genetically modified cell strains (and typically parent strains as well); (b) analyzing the images with one or more algorithms that provide quantitative representations of the phenotypes; and (c) comparing the quantitative representations of the phenotypes with (i) each other, (ii) the parent strain, or (iii) a quantitative representation of a phenotype of a cell that is genetically similar or identical to one or more of the cell strains.

Preferably, the genetically modified cell strains are deletion mutants having one or more genes deleted from the genome of the parent strain. Each of the deletion mutants may lack a single gene present in the parent strain. In a specific embodiment, the collection of genetically modified cell strains includes the deletion mutants provided by the Saccharomyces cerevisiae Deletion Consortium. In such collection, the genetically modified cell strains may include mutant strains having modified, but not deleted, essential genes of Saccharomyces cerevisiae.

The phenotype images may be generated in various manners. Often it will be desirable to highlight certain cellular features by marking those features. Thus, the above method may also include the following: (i) marking one or more cell features of the genetically modified cell strains and/or parent strains so that said features can be highlighted in the images of the phenotypes; and (ii) imaging the genetically modified cell strains to produce the images of the phenotypes, wherein the cell features are highlighted in the images of the phenotypes. In one preferred embodiment, the genetically modified cell strains are yeast strains and that are stained with a first stain for the cell wall, a second stain for the genetic material, and a third stain for the cytoskeleton. In a specific embodiment, the first stain is concanavalin A, the second stain is DAPI, and the third stain is rhodamine phalloidin.

The image analysis component of this invention may take various forms. In one preferred embodiment, it involves the following: (a) receiving the intensity versus position data from one or more markers on the parent and/or genetically modified cell strains; (b) quantifying geometrical information about said markers; and (c) quantifying biological information about the genetically modified cell strains. Preferably, the quantitative representations of the phenotypes include one or both of the geometrical information and the biological information.

Comparing the quantitative representations of the phenotypes can help classify and understand the actions of various genes and environmental influences. In one embodiment, comparing the quantitative representations of the phenotypes involves comparing the quantitative representations of the phenotypes with each other in order to cluster the phenotypes and identify common functional traits shared between multiple genetic modifications. Alternatively, the comparison compares a quantitative representation of a phenotype of one or more of the cell strains with a quantitative representation of the phenotype of a genetically similar or identical cell that has been treated with a drug or a drug candidate.

The quantitative phenotypes of this invention may be stored in a database including records identifying the phenotypes and the quantitative representations of the phenotypes. Such database may be linked with another database containing non-morphological information (e.g., gene expression data) about the collection of genetically modified cell strains or other strains.

Another aspect of the invention pertains to computer program products including a machine-readable medium on which is provided program instructions, data structures, databases and the like for implementing a method as described above. Any of the methods of this invention may be represented as program instructions that can be provided on such computer readable media.

These and other features and advantages of the present invention will be described below with reference to the associated drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a process flow diagram depicting a sequence of operations that may be employed to generate quantitative phenotypes for a collection of congenic strains.

FIG. 2 is a process flow diagram depicting a sequence of operations that may be employed to prepare cells for imaging in accordance with an embodiment of this invention.

FIG. 3 is a schematic illustration of the yeast cell division cycle.

FIG. 4 is a series of images taken for a yeast cell at various stages in the cell division cycle; the nucleus (blue), actin (red), and cell wall (green) are highlighted by virtue of their fluorescence in these images.

FIG. 5 is a schematic illustration of the actin distribution within a yeast cell at various stages of the cell division cycle.

FIG. 6 presents a series of images showing actin and mictrotubule distribution in budding yeast.

FIG. 7A presents images of yeast cells that have been exposed to benomyl and other yeast cells that have not been so exposed; the cells have been stained to highlight cell walls and nuclei.

FIG. 7B graphically presents the data from FIG. 7A, showing intensity distribution versus position graphs for the cell wall and the nuclei.

FIG. 8 presents three separate images of yeast cells, with one highlighting the cell walls, another highlighting the actin, and a third highlighting the nuclei. Associated graphs show how these three components distribute themselves with respect to one another in polarized and unpolarized yeast cells.

FIG. 9 is an image of yeast cells stained with calcofluor white to highlight scars left on mother cells from earlier buds.

FIG. 10 is an image of yeast cells undergoing constitutive pheromone response and having a characteristic morphology.

FIG. 11 presents a series of images highlighting actin in yeast cells and illustrating actin derangement in mutant Saccharomyces cerevisiae.

FIG. 12 presents a series of images illustrating the morphology and nuclear position of yeast morphological mutants having abnormal buds and abnormal nuclear position.

### DESCRIPTION OF THE PREFERRED EMBODIMENTS

As mentioned, the Saccharomyces cerevisiae Deletion Consortium has created a complete set of deletion strains. These strains are congenic to a single parent known as BY4743. In other words, each strain differs from the parent by only a single gene. Each strain is a perfect deletion, in that the deleted gene is removed starting with the initiating methionine and ending with the stop codon. In other words, the entire open reading frame is deleted. While this invention will be described in the context of phenotyping the yeast strains from the Consortium, the ideas presented herein could easily be extended to other Saccharomyces strains or other organisms or collections of organisms in which various deletion strains are available or become available, such as the human pathogen, Candida albicans.

Yeast is convenient because it is a very genetically tractable organism, it is easily cultivated, and a high percentage of its genes have homologs in humans. The Saccharomyces cerevisiae Deletion Consortium is centered at Stanford University, Stanford, Calif., where double stranded DNA deletion cassettes constructs for the deletion are created. More information about the Saccharomyces cerevisiae Deletion Consortium and the strains it has created can be found at http://sequence-www.stanford.edu/group/yeast deletion project/. The genome for Candida albicans has recently been completely sequenced. To the extent that the following discussion specifies Saccharomyces cerevisiae, it could equally apply to Candida albicans.

Because the individual strains made by the Deletion Consortium contain perfect deletions, one can precisely measure how a given gene influences an organism's phenotype in accordance with this invention. A comparison of the phenotype of the parent strain and a deletion strain provides valuable information about the gene's function. It also allows one to characterize new phenotypes based on their similarity to known phenotypes of known deletion strains.

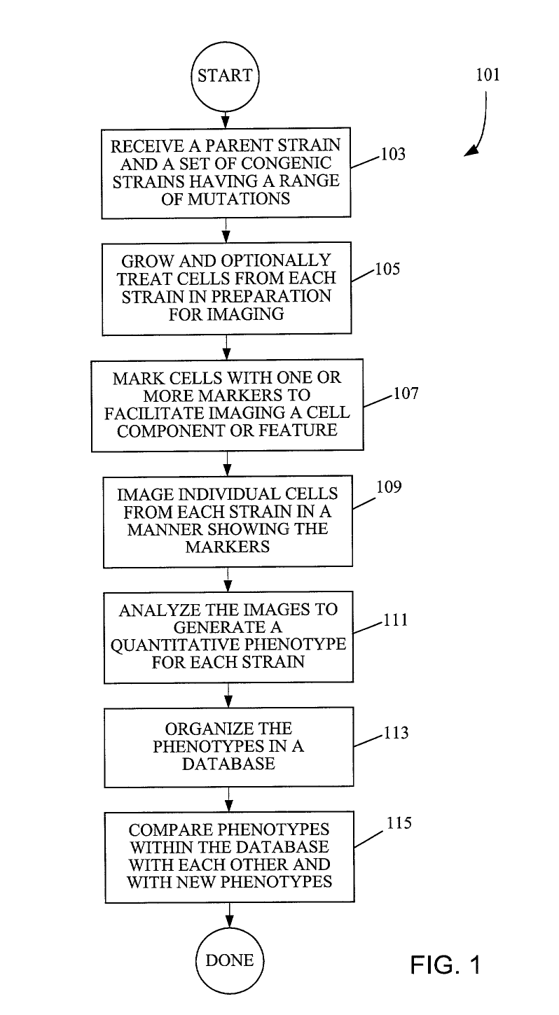

FIG. 1 presents a sample process 101 flow that may be employed in the context of the present invention. Process 101 begins with receipt of a congenic set of strains having a range of mutations. See 103. In a preferred embodiment described herein, the congenic set of strains is the complete set of deletion strains obtained from the Saccharomyces cerevisiae Deletion Consortium. The strains to be used include haploid deletion mutants (both a and alpha mating types) heterozygous diploids and homozygous diploids. For the case of essential genes, one may augment the Deletion Consortium mutants with insertion mutants that are viable or heterozygous diploids.

After receiving the complete set of congenic strains, each strain must be separately prepared for imaging and analysis. See 105. Generally, the cells must be grown and incubated. In some cases, the cells will simply be grown without any particular environmental stresses. In other instances, the cells will be exposed to a particular environmental stress such as a drug or toxin. Of course, combinations of stresses may also be employed.

Some cellular features can be contrasted from the remainder of the cell by specific markers. As described more fully below, some markers are chosen to contrast the entire cell, the cell organelles, and other markers are chosen to contrast specific biomolecules. Block 107 depicts the marking operation in FIG. 1. Often, the process will simultaneously treat the cells of a strain with a collection of different markers, each contrasting a different aspect of the cell.

After the cells to be imaged have been optionally marked at 107, an imaging system images the wells in which they were plated in a manner that highlights the cell markers. See 109. Thus, for example, some images may clearly show the cell walls, while other images clearly show the nuclei, and still other images show the actin cytoskeleton. Imaging systems useful for this purpose will be briefly described in more detail below.

Next, the process analyzes the individual images to generate a quantitative phenotype for each strain. See 111. Typically, the phenotype is defined by a combination of features extracted computationally from collected images. Examples of such features include the shape and size of cellular organelles, the shape and size of the cell wall or cell membrane, and the location of biomolecules and cellular organelles within the cell. Each of these features may be represented as a numeric value or combination of numbers. In some embodiments, each phenotyping is represented by a combination of such numeric values organized as a “fingerprint.”

The phenotypes generated in this manner are optionally stored in a phenotype database at 113. Regardless of how the phenotypes are stored and organized, they are used for comparison to other numerically represented phenotypes. See 115. This comparison may involve looking for similarities between phenotypes already stored in the database. Alternatively, the comparison may involve matching phenotypes of unknown strains with phenotypes of known strains stored in the database. Determining a distance between two separate phenotypes indicates how closely related those phenotypes may be and thus allows prediction of gene function.

In the specific embodiment described herein, the various mutant yeast strains from the Saccharomyces cerevisiae Deletion Consortium are phenotyped. These strains are produced by “surgically” deleting one copy of the gene in a diploid cell by virtue of mitotic recombination of a selectable marker gene flanked by DNA sequences that define the start and stop of the open reading frame. The resulting heterozygous cell is then sporulated to produce a haploid deletion strain. By mating two haploid strains, each lacking the gene of interest, one produces a desired homozygous deletion diploid cell. The complete deletion set therefore contains heterozygotes, homozygous diploids, and haploid deletions of both a and alpha mating types, comprising approximately 21,800 strains (allowing for essential genes). For sporulation defective mutants, direct deletion of the gene was performed on haploids.

For most strains, images show phenotypes of live strains; that is, viable deletion mutants. As mentioned, however, about 17 percent of the approximately 6200 genes of Saccharomyces cerevisiae are essential to the organism's survival. To the extent that a yeast mutant lacking an essential gene can be created, such mutants cannot be imaged live. Nevertheless, it would be desirable to show how each essential gene influences a live cell's phenotype. In one embodiment, strains are created in which essential genes are modified, rather than deleted. Some such mutants provide live cells having modified phenotypes. In one embodiment, for essential genes, heterozygous diploids as well as the insertion mutants are used. The heterozygous diploids include one normal copy of the essential gene and one abnormal copy of that gene. The abnormal copy may have a completely deleted or highly mutated gene. In a specific example, the insertion mutants for essential genes were created by Michael Snyder of Yale University. These mutants are described at http://ygac.med.vale.edu/. In these examples, the essential gene mutants are analyzed and used in accordance with this invention to provide phenotypes of living cells having defective essential genes.

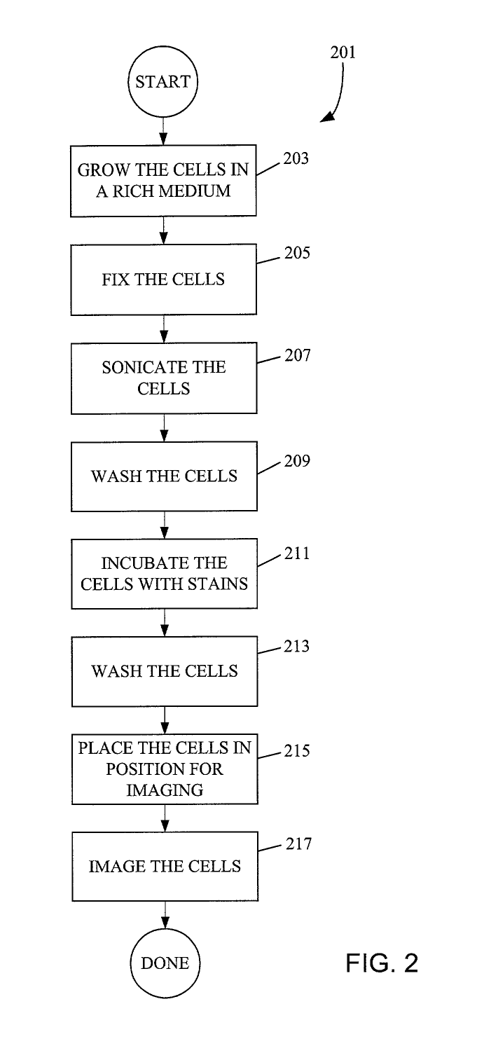

After the relevant strains or cell lines have been selected, each individual strain or cell line must be prepared for separate imaging. FIG. 2 presents an example of a process 201 for preparing a single strain or cell line for imaging. Preferably, this process is performed in a high-throughput automated manner, possibly with the aid of a robot. The process begins at 203, where the cells of the selected strain are grown in a rich medium (e.g., YPD). In some instances, the cells are grown in this medium without environmental stress. For the deletion strains used in a preferred embodiment of this invention, examples of preferred media include YPD (Adams et al. 1997, Methods in Yeast Genetics, Cold Spring Harbor Laboratory Press, incorporated herein by reference for all purposes). In this embodiment, the cells are grown at 30 degrees Centigrade. After the cells have been grown for a defined period (e.g., 3 population doublings), they are fixed at 205. Various agents may be used to fix cells prior to imaging. In a specific embodiment of this invention, 2-5% formaldehyde is used to fix the cells.

Certain cells such as yeast cells have a propensity to aggregate or “clump.” Clumped cells are difficult to analyze with image analysis software because they may appear to be one large cell. And even if the software can identify multiple cells within a “clump,” it may have difficulty identifying specific features within individual cells of the clump. Therefore, the process should include an operation which reduces the likelihood that cells will clump. To this end, process 201 optionally requires that the cells be sonicated. See 207. Note that if the cells are sonicated, this procedure may be performed either before or after the cells have been fixed. Various tools may be used to sonicate the cells. For example, a water bath sonicator will sonicate the individual cells of a plate that floated in the water bath sonicator. An example of a suitable sonicator is the Branson Ultrasonic cleaner available from Branson Ultrasonics, Danbury, Conn. Alternatively, a probe sonicator can be used prior to plating cells. An example of a suitable sonicator for this purpose is the Branson Sonifier available from Branson Ultrasonics, Danbury, Conn. Another suitable system, the XL-2020 Microplate Sonicator available from Misonix, Inc. of Farmingdale, N.Y., sonicates individual 96 well plates.

After the cells have been optionally sonicated, they are washed at 209. Next, the cells are incubated with the selected stains at 211. Examples of suitable fluorescent stains will be described in detail below. For now, simply recognize that the stains are selected to highlight particular cell markers for subsequent imaging. Next, the stained cells are washed at 213. The washed cells are then placed in position for imaging. See 215. Finally, the cells are imaged at 217. Preferably, the various stains are applied simultaneously in order to improve the process throughput. Note that a technology for processing large quantities of cells in a high throughput manner is described in U.S. patent application Ser. No. 09/310,879 by Vaisberg et al.; U.S. patent application Ser. No. 09/311,996 by Vaisberg et al.; and U.S. patent application Ser. No. 09/311,890 by Vaisberg et al., each of which is incorporated herein by reference for all purposes.

To provide baseline images, each deletion mutant and parent strain is imaged without environmental stress. However, additional phenotypic information can be obtained from combinations of deletions and environmental stresses. Most such stresses are introduced while the cell is growing at 203 in process 201. Examples of such stresses include high temperatures (e.g., between about 34 and 42 degrees Centigrade), low temperature (e.g., between about 10 and 20 degrees Centigrade), high salt concentration (e.g., between about 0.5 M and 1 M ionic species in the media), and the presence of specific chemical agents. A few specific examples of salts that can provide interesting results include sodium chloride, lithium chloride, calcium salts, and manganese salts. Examples of other interesting stress inducing conditions include using minimal quantities of media and nitrogen starvation. Examples of chemical agents include toxins, suspected toxins, drugs, and drug candidates. From a more specific biochemical perspective, examples of chemical agents include pheromones, actin depolymerization agents, and microtubule depolymerization agents. In a specific example, yeast cells are treated with α-factor, a mating pheromone for yeast. In another specific example, yeast cells are treated with benomyl, a compound that depolymerizes microtubules in cells. Other examples include antifungal drugs including azoles, 5-fluorocytosine, griseofulvin, terbinafine, and amphotericin B. Each of these different stresses produces a separate phenotypic fingerprint generated by imaging the associated cells and quantifying features in those images.

As mentioned in the discussion of FIG. 2, the cells may be marked to emphasize certain features. Selection of appropriate markers requires balancing certain considerations. First, a marker should be chosen to highlight an interesting, informative feature of the cells. For example, a marker may highlight a cell wall or cell membrane, a sub-cellular organelle, or a cellular biomolecule. Second, a marker should not significantly interfere with the cellular phenotype. In preferred embodiments, for example, yeast markers should be able to penetrate the cell wall without damaging it. If one must modify the cell wall, the phenotype will contain artificial features. For this reason, it is preferred that non-immunological markers be used to mark yeast cell features. Antibodies and antibody components are too large to pass through the yeast cell wall without having first modified the cell wall. Another consideration in selecting markers is the ease with which they may be applied to yeast cells (preferably fixed yeast cells in suspension or living yeast cells in suspension).

Examples of sub-cellular organelles that may be marked include the nucleus, the mitochondrion, the Golgi, lysosomes, peroxisomes, the endoplasmic reticulum, vacuoles, etc. Examples of cellular biomolecules that may be marked include nucleic acids, cytoskeleton proteins, glycoproteins, chitin, cytoskeletal motors, etc.

Some specific examples of markers include DAPI (for DNA), fluorescent concanavalin A (for the cell wall and overall cell shape), rhodamine phalloidin (for actin cables and patches), Calcofluor White (for chitin deposited at bud scars) and a variety of fluorescent stains for the endoplasmic reticulum, mitochondria, lysosome and vacuole. For subcellular organelles such as the mitochondria, endoplasmic reticulum, lysosome and vacuole, fluorescent markers exist that mark each of these organelles based on differences in membrane potential. Use of these markers will allow for a “live fingerprint” as well as the fixed fingerprint described below.

In a specific embodiment, three separate cell markers are stained in a single operation. The markers are for labeling the cell wall, DNA, and actin. In one example, the cell wall is stained with concanavalin A (conA), DNA is stained with DAPI, and actin is stained with rhodamine phalloidin. All three of these may be applied to the cells in a single operation.

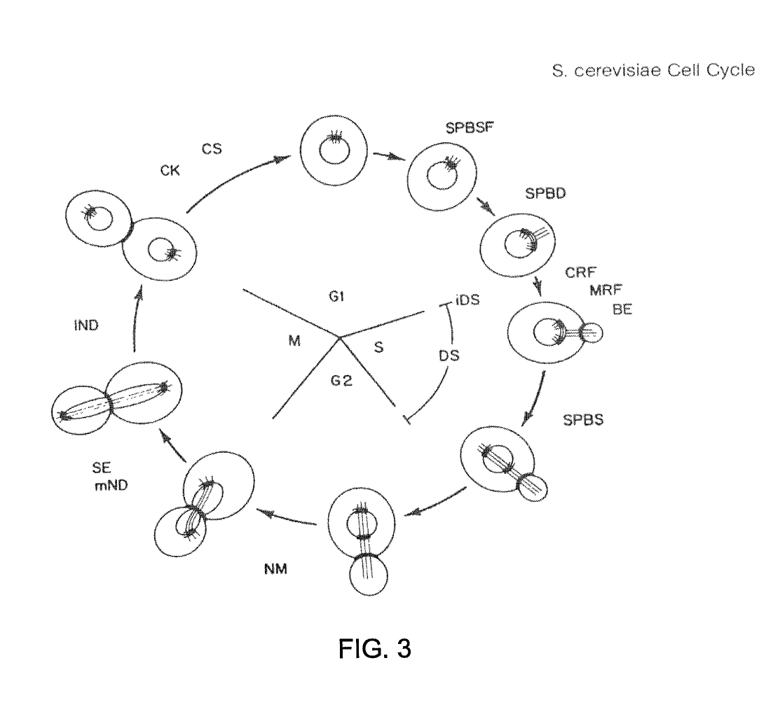

In yeast, the shape of the cell wall is very informative. Rather gross shape changes specifically indicate where the cell currently resides in the overall cell cycle. This is illustrated by the Saccharomyces cerevisiae cell cycle illustrated in FIG. 3. This figure is taken from Hartwell 1981, “The Molecular Biology of the Yeast Saccharomyces cerevisiae,” Pringle J. R. and Hartwell, L. M., pp. 97-142, Cold Spring Harbor Laboratory Press, incorporated herein by reference. Deviations from expected cell shape are easy to detect, and significantly, a large number (at least 50) of these deviations correlate with genetic changes in the yeast genome.

The location and concentration of DNA can indicate the cell cycle stage and can identify certain mutants that mislocalize their nuclei. Such mutants can be classified using the DNA stain. The location and arrangement of actin can also provide valuable information about the cell. Actin proteins organize themselves into two distinct structures: cables and patches. The structures are arranged in certain orientations depending upon the “polarization” of the cell. Polarization in yeast cells indicates certain cell events such as bud emergence and generation of the mating projection. Bud emergence begins in the S Phase of the cell cycle as indicated in FIG. 3.

To provide an example of how the three preferred stains work together, consider the normal budding of a vegetatively growing yeast cell. Initially, a bud begins to form on a side of the cell wall. This can be easily seen in cells stained with conA. Next, the nucleus moves to the bud neck and divides. This can be easily seen in cells stained with the DAPI DNA stain. In addition, during budding, the actin polarizes. Specifically, the cables and patches arrange themselves to point toward the incipient bud. The stained actin facilitates visualization of this process. In abnormal cells, this budding process can exhibit numerous variations. For example, the bud may form but the nucleus does not enter it. In such cases, the actin may be either polarized or unpolarized, depending upon the type of abnormality. Furthermore the actin state mirrors the molecular state of a class of cell cycle control molecules, the cyclins (see 1995, Lew, D. J. and Reed, S. I., “Cell Cycle Control of Morphogenesis in Budding Yeast,” Curr. Opin. in Genetics and Development, 5: 17-23, incorporated herein by reference).

Obviously, the combination of these three markers provides a rich source of information about the cell's state and its deviation from normality. These markers, alone or in combination with other markers, can be quantified and combined to provide phenotypic fingerprints for each deletion mutant.

Considering FIG. 3, the outer shape of the cell in its various stages represents the cell wall. The inner circle or oval represents the cell nucleus. The nucleus will be highlighted by DNA stains. The distinct orthogonal lines on the nucleus represent microtubules. These are typically marked with immunological markers. Unfortunately, introduction of such markers requires disruption of the cell wall. Alternatively, the microtubules (or many other proteins and/or structures for that matter) can be marked with a green fluorescent protein analog. In the case of GFP-marked microtubules, the cell expresses a GFP-tubulin fusion protein.

To analyze the microtubule cytoskeleton, one may mate all haploid deletion mutants (and haploid insertion mutants in essential genes) with a haploid strain of the opposite mating type that expresses a GFP-tubulin fusion protein, enabling visualization of microtubules in live or fixed cells. Alternatively one could introduce the GFP fusion proteins by transformation. This procedure can be carried out en masse, by printing both strains in a 96-well format.

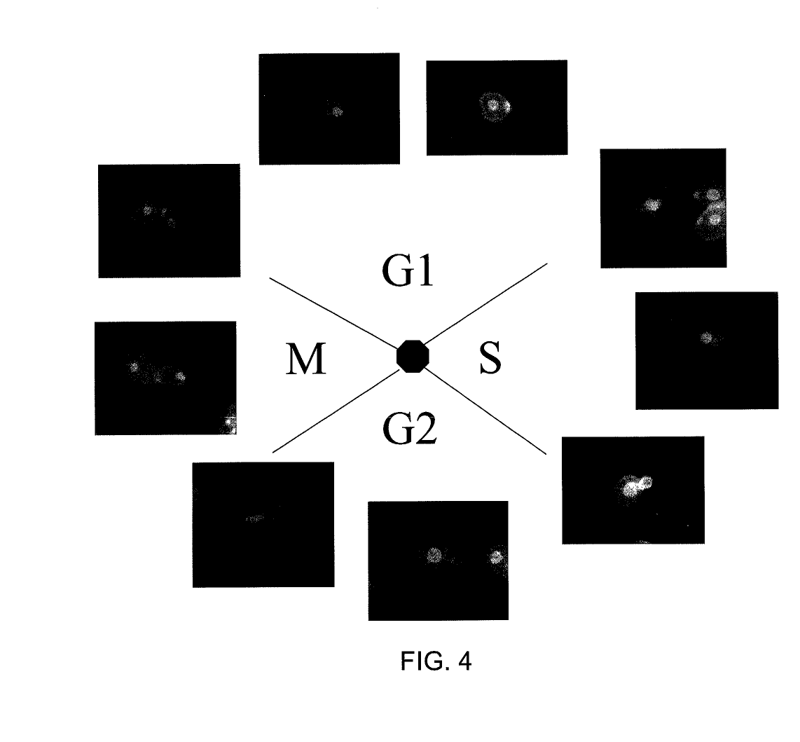

FIG. 4 presents images of normal Saccharomyces cerevisiae cells marked with each of the three stains mentioned above. The concentrated blue regions represent DAPI stained nuclei. The red regions represent rhodamine phalloidin stained actin. And the green edges represent conA stained cell walls. From these images, one can see how the cell wall, the nucleus, and the actin change during the cell cycle of a normal yeast cell. Deviations from these normal markings can be correlated with changes to the yeast genome such as deletions of a single gene. These differences can be quantified and provided in a fingerprint for each strain.

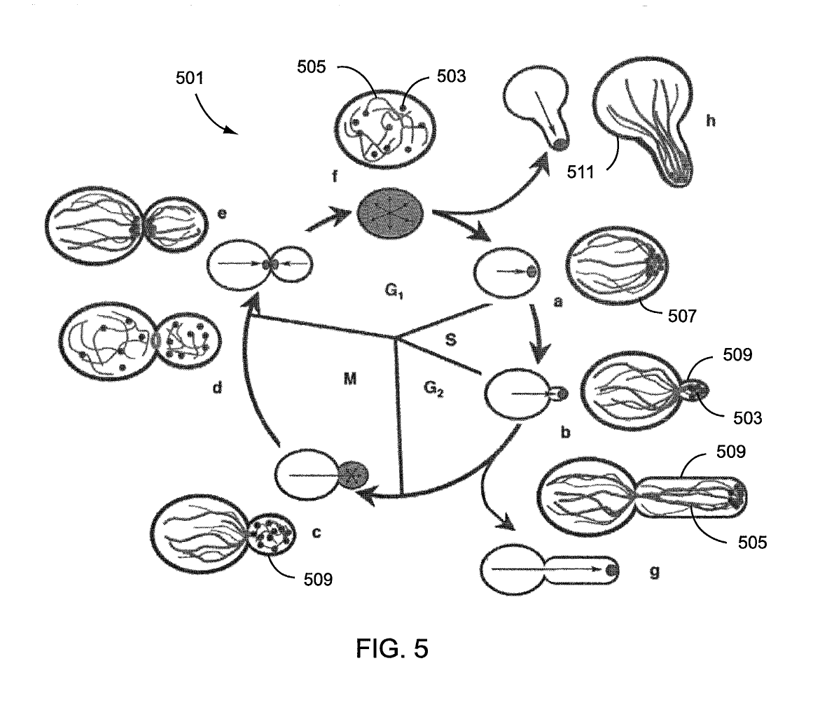

FIG. 5 illustrates how actin is distributed within a given cell during different phases in the cell cycle. The overall cell cycle, represented by 501, is divided into the G1 phase, the S phase, the G2 phase, and the M phase. A cell 502 in the G1 phase contains actin in two forms: patches 503 and cables 505. As the cell enters the S phase, its actin becomes polarized as illustrated in the cell state 507. As the cell continues through the S phase (indicated by state a), the bud 509 begins to form. The patches 503 concentrate in the bud. In the G2 phase, actin cables 505 form in an elongated bud 509. As the cell enters its M phase (indicated by state a), some actin patches 503 and cables 505 form in cells within bud 509. As mitosis proceeds, the actin cables and patches rearrange themselves within the two daughter cells as illustrated in the cell states d and e. While in the G1 phase, the cell may mate with another cell of the opposite mating type. The yeast cell that is ready for mating develops a projection 511 as illustrated in cell state h. The actin within the cell rearranges as shown.

In order to obtain the relevant marker information from the stained cells, the cells must be imaged by an appropriate method. Various imaging techniques are available to meet this requirement. Many markers emit photons of a specific wavelength after excitation with light of a marker-specific excitation wavelength. The imaging system should be tuned to detect such wavelengths. Examples of suitable imaging systems are presented in U.S. patent application Ser. Nos. 09/310,879, 09/311,996, and 09/311,890, previously incorporated by reference.

Given the relatively small size of yeast cells, they are preferably imaged at a magnification of between about 200× and 400×, requiring the use of 20× and 40× objectives, respectively, in combination with a 10× photo ocular. In addition, the imaging system should be designed to auto-focus on cells at that magnification level. Further, because yeast cells do not adhere well to plastic substrates, the plates on which they are to be imaged should be coated with an adherent material such as polylysine.

Image analysis involves quantifying or otherwise characterizing an image of a cell to produce a phenotypic fingerprint or other representation. Image analysis is preferably performed in whole or part by image processing software and/or hardware. An example of a suitable hardware system is presented in the above mentioned U.S. patent application Ser. Nos. 09/310,879, 09/311,996, and 09/311,890.

Image analysis may also include some preprocessing such as filtering to remove “clumped” cells from consideration. Clumped cells are easily identifiable by their relatively large size and/or atypical shapes. Software that recognizes such clumps can be used to separate the clumped and unclumped yeast cells in an image.

Inputs to the image analysis component of this invention include the location and “intensity” (usually representing concentration) of various cell markers that can be detected by the image analysis procedure. For example, in the preferred embodiment described herein, the location and intensity of markers for the cell wall, DNA, and actin serve as inputs. The intensity can be presented as a local intensity or an intensity averaged over multiple areas. For example, the intensity may be averaged over a few pixels, a particular organelle, or the entire cell. Using two-dimensional coordinates, one can identify the shapes and sizes of various organelles or cells.

One somewhat useful program for quantifying cellular features is “Metamorph” available from Universal Imaging Corporation of Westchester, Pa. In this product, a user picks a particular cell or field of cells and then selects a particular parameter or routine to use for his or her analysis. In one specific example, this program was used to identify large budded yeast cells within a group of yeast cells and clumps appearing in a single image. The budded cells were identified based upon the measured length of the cells.

In one example, the following routines from the Metamorph software were used.

MetaMorph Image Analysis

ConA (cell wall):

1. Scale image to 8 bit under Process, Scale 16 bit image.

2. Low Pass under Process, to smooth out the edges of the objects.

3. Threshold image until the object is highly contrasted against the background.

4. Open Integrated Morphology Analysis under Measurement.

5. Measure area, fiber length, and shape factor by selecting objects of interest. Do not include clusters or clumps.

6. Save State to save the filter parameters so it can be used to analyze different sets of images.

DAPI (DNA):

1. Perform steps 1 to 4 from ConA analysis.

2. Load State to load the saved parameters. Only unclustered objects are highlighted after this step is performed.

3. Select LineScan tool under Measurement.

4. Select LineTool from tool box.

5. Point and drag from on end of the object to the other end and release mouse. Several parallel lines should appear along the long axis of your object of interest.

6. The plot in the LineScan window will show the intensity distribution. We can classify budded cells using this tool.

7. SaveState, so that the filter parameter can be used again to analyze other images.

Rhodamine phalloidin (actin):

Analysis of actin is the same as DAPI except that one is measuring the actin intensity instead of DNA intensity. We can classify mutants according to the localization of the actin filaments and patches.

From a purely geometric perspective, the image analysis outputs include the cell's shape and size. For the nucleus, the geometric outputs may include the nucleus' shape, size, number, intensity, and position within the cell. At certain stages within the cell division cycle, one expects to find two nuclei. If an unexpected number of nuclei are found in any cell, one can assume that it is abnormal in some respect. For actin, the geometric outputs may include the actin's distribution, orientation, morphology, concentration, and location within the cell.

At a quantitative/fingerprint level, the image analysis outputs include the deviation of above parameters from values expected for a normal cell. Further, these deviations are specific for the cell's position in the overall cell cycle.

From a biological perspective, the image analysis output may specify where in the cell cycle a particular cell resides and whether it is abnormal with respect to its congenic parent. From the perspective of the cell wall, the biological outputs may specify whether the cell is budding, how is it budding, where it is budding, the size of the bud, whether the cell is ready to mate, what its size is with respect to its parent, etc. For the nucleus, relevant biological outputs include whether the cell's nucleus is located at an expected position, whether the cell contains the correct number of nuclei, whether the DNA is concentrated in the nucleus as expected as well as the DNA replication state, etc. For actin, relevant biological outputs include the degree of actin polarization, how diffuse the actin is arranged (smooth versus granular patches), whether the actin forms “aggregates,” whether it forms “bars,” etc.

For each of these biological parameters, the image analysis process will apply a numeric value. This provides a much-improved representation of phenotype in comparison to conventional visualization and verbal qualitative characterization. Note that this invention also allows a very fine segmentation between cell division cycle steps. In other words, the algorithmic characterization places the cell at a very precise location within the overall cell cycle—effectively subdividing the traditional cell cycle classes into multiple subclasses.

In one example, the image processing operations of this invention determine whether actin bars or actin aggregates are formed and where they are located within the cell. Derangements of actin distribution may appear in some deletion mutants or environmentally stressed cells adding quantitative information to a strain's “fingerprint.”

In one preferred embodiment, cells are profiled based on the following four elements: cytoskeleton, cell morphology, organelles, and DNA replication state. The DNA replication state may be identified by using DAPI as a marker; if the DNA is being replicated, the DAPI intensity will be up to twice as great compared to cells that have not replicated their DNA. The cell morphology may be marked with conA, which binds to the cell wall. The nucleus and mitochondria are imaged with DAPI. The cytoskeleton may be marked with rhodamine phalloidin, which binds to actin.

Various algorithms may be employed to obtain the necessary information. Examples include statistical classifiers of various sorts, including image segmentation, morphological measurements, texture analysis, frequency analysis, wavelet decomposition, digital wavelet transformation, and the like. Preferably, the algorithms operate on a cell-by-cell basis. In other words, the image analysis process should be able to analyze each cell independently. This is often necessary because the individual cells have asynchronous cell cycles. Meaningful phenotype information may be enhanced by first properly identifying a cell's position in the cell division cycle.

In one approach, a cell-by-cell analysis involves three operations: segmentation, feature extraction and statistical analysis. For example, cell cycle is determined from DAPI images of mammalian cells in the following steps. First, the nuclei are segmented. That is, the pixels that make up each nucleus are identified. This may be done by either edge detection or thresholding. Second, the total feature intensity is computed. Total intensity is the sum of the pixel intensities in each nucleus and is a surrogate measure of DNA content. A histogram of the total intensity for all cells in the image will appear as a mixture of three normal distributions corresponding to G1, S and G2. A statistical procedure called the EM algorithm (Expectation-Maximization) may be used to classify cells into G1, S or G2. Proportions of G1, S and G2 cells are also computed. The algorithm may also identifies mitotic cells. For more details of such process, see U.S. patent application Ser. No. 09729,754 filed Dec. 4, 2000, naming Vaisberg et al. as inventors.

Yeast cells may be classified by their cell shape as determined by, for example, the conA marker of the cell wall. There are four principal categories of wild type cell shape (with numerous subcategories): oblong, oblong with small bud, oblong with medium bud and oblong with large bud. A cell-by-cell approach may be used in which cells will be segmented and features computed. Features for shape representation and description is a rich field in image analysis. Many feature analysis routines are possible, including: Fourier transforms, Hough transforms and a graphical representation based on region skeleton. One challenge in this analysis is that cells may clump together making it difficult to determine if two adjacent cells are mother-daughter cells or are unrelated. Information from the other two marker images may be used to discriminate clumped cells as may thresholding of the entire field of cells. In fact, such a “clumping algorithm” serves two purposes, 1) to eliminate cell aggregates from cell by cell analysis and 2) to identify those mutants that exacerbate clumping as part of their phenotype. The phalloidin marker identifies the actin within a cell and hence the cell's polarity. A cell's polarity is just one example of many features that can be computed from overlaying images.

The outputs from image analysis are preferably organized into specific data structures (e.g., fingerprints or groups of fingerprints) for each cell. For example, a given deletion mutant may have a first phenotypic fingerprint for normal growth conditions (e.g., rich media at 30 degrees Centigrade as mentioned above), a second phenotypic fingerprint for growth at elevated temperatures, a third phenotypic fingerprint for growth in highly saline conditions, a fourth phenotypic fingerprint for exposure to a particular drug, etc. Remember that the fingerprints are comprised of various quantitative values (e.g., the cell is in cell cycle phase n and has an actin polarization of x microns) and possibly some yes/no characterizations (e.g., the cell is ready to mate). In some embodiments, each genetically pure strain has a single composite fingerprint comprised of information from a variety of environmental conditions. The fingerprint may be viewed as a vector comprised of several scalar values. For certain phenotypic comparisons, these scalar values may be weighted differently.

Preferably, the information about each phenotype is stored in a database or “knowledge base.” The phenotype information may be organized within such database in a variety of ways. In one embodiment, each cell image presents a unique record. Preferably, each unique combination of genotype and environmental conditioning is uniquely identified. The fingerprint or other quantitative representation of a phenotype is stored in the data record or at least pointed to by the record. The data records may also specify a deviation of the phenotype at issue from its congenic parent. The deviation may have a numeric value (e.g., an average, a weighted average, a Euclidean distance, etc.). Still further, the database records may identify how the cells under consideration are grouped. A group of phenotypically related cells is referred to herein as a cluster.

In one example, each deletion mutant is given a unique phenotypic fingerprint. Those phenotypes are compared with each other using an appropriate algorithm that makes biologically relevant comparisons between the fingerprints of individual mutants. Those phenotypes that are deemed close to one another by the algorithm are grouped in the same cluster. All phenotypes in a cluster presumably have a similar function. Examples of functional clusters include actin/actin binding proteins, cell wall proteins, cell cycle control proteins, and mating response proteins. Examples of gene classes from the Saccharomyces Genome Database (http://genome-www.stanford.edu/saccharomyces/) that are involved in these cellular processes include the following:

Cell wall- CBK, CCW, SCW, WSC
Actin-ABP, ACT, AIP, ANC, ARK, ARP, CAP, CRN, DAD, DIP, FIP, FIR,
GIP, HIF, IMP, KRI, LIF, NIF, PIP, SAC, SIP, TCI, TWF, VTI, YIF
Cell cycle- CDC, CDH, CEF, CKS, HOF, LSD, NRF, SCH, SDC, SYF, TFS

In one example, there is a deletion mutant lacking a gene of unknown function. For this mutant, the process generates a phenotypic fingerprint specifying that its bud is 10% smaller than normal and that its actin is 60% polarized and 40% diffuse. Normally, one could not detect these features in a simple analysis by eye. From this information, one could conclude that the gene is involved in the processes that generate daughter cells and polarize actin. However, because its deletion did not entirely arrest the processes, one could also conclude that the gene is not a “prime mover” in the processes under the examined conditions. Possibly, that gene is part of a large protein complex that is responsible for ensuring that the daughter is the right size and the actin is polarized. But in its absence, the protein assembly that it is normally a part of can still function, but in a less effective manner. If the gene was present, then the daughter cell would be of normal size and the actin polarization would be 100%. If the gene is a prime mover in the process, it would totally prevent polarization of actin and/or generation of the daughter cell. By determining which parts of a larger process the gene affects, the phenotype fingerprint can also be used to determine where in a cellular process pathway the gene operates. Some genes participate in multiple cellular pathways. Such genes will sometimes be identifiable by virtue of their clustering in two or more groups.

To the extent that the quantitative phenotypes of this invention are provided in a database or are otherwise organized in a logical convenient manner, they may be linked to other databases containing data characterizing yeast (or other organism of interest). For example, mutants from the Deletion Consortium (or other mutant collection) are being analyzed and cataloged based on expression patterns (mRNA levels), protein-protein interactions, growth defects, localization of proteins within the yeast, etc. As this information is organized and stored in databases, it will be useful to link or integrate the phenotype data of this invention with the data from these other projects. Thus, for a particular gene, one could query a collection of databases to get many pieces of relevant and related information about that gene.

In one embodiment, the database is organized to provide phenotypic fingerprints for each strain in the Deletion Consortium Collection. Each strain is associated with a set of downloadable images and descriptive information regarding the specific features extracted for each marker. Additionally, phenotypes of individual strains may be clustered with similar phenotypes.

Yeasts (including Saccharomyces and Candida) are a subset of fungi. Importantly, both yeasts and fungi can manifest as human pathogens, often resulting in debilitating disease states or death. The techniques described here can be applied to any species of yeast or fungus for which mutants are available. Furthermore, in the absence of gene deletions (or in combination with such mutants) the technique described here can be used to profile the effects of a variety of drugs that have antifungal properties. In this manner the chemical phenotype, alone or combined with our genetic fingerprint can be used to classify the mechanism of action of antifungal drugs as well as to determine the gene product that is the target of such agents.

### EXAMPLES

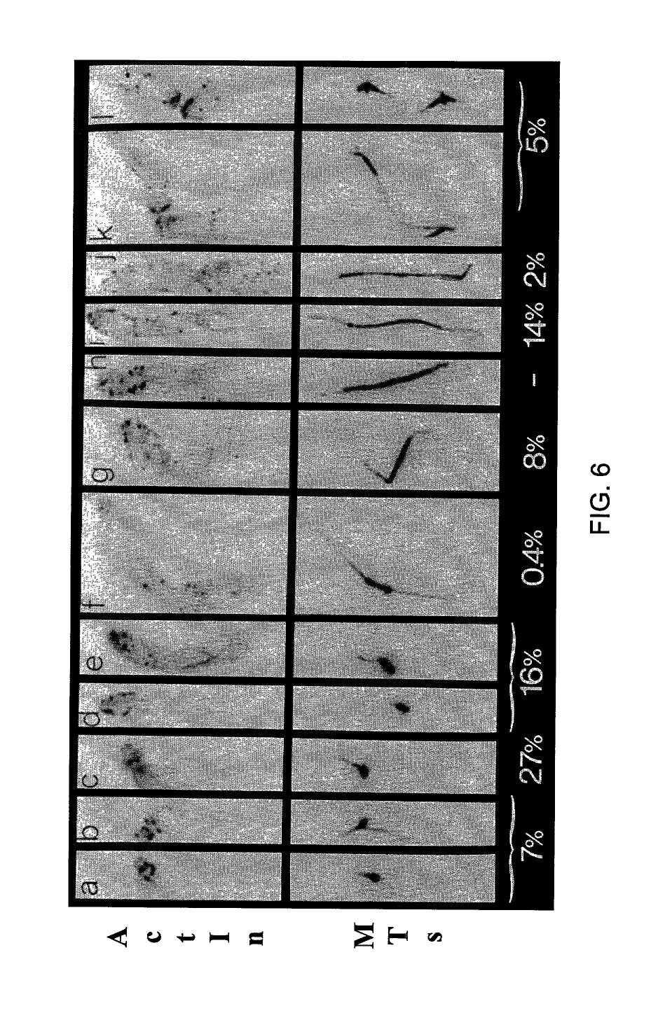

FIG. 6 shows images of actin and tubulin distribution in budding yeast. Each vertical pair of images corresponds to the same phase of the yeast cell's budding process. In this figure, the numerical legend at the bottom refers to the fraction of cells in the population at a given stage of the cell cycle. The actin was marked with rhodamine phalloidin and the tubulin was marked with an anti-tubulin antibody. The immunofluorescence was imaged. The phenotypic information that can be derived from these images includes the state of the mitotic spindle, as well as the cells position within the cell cycle.

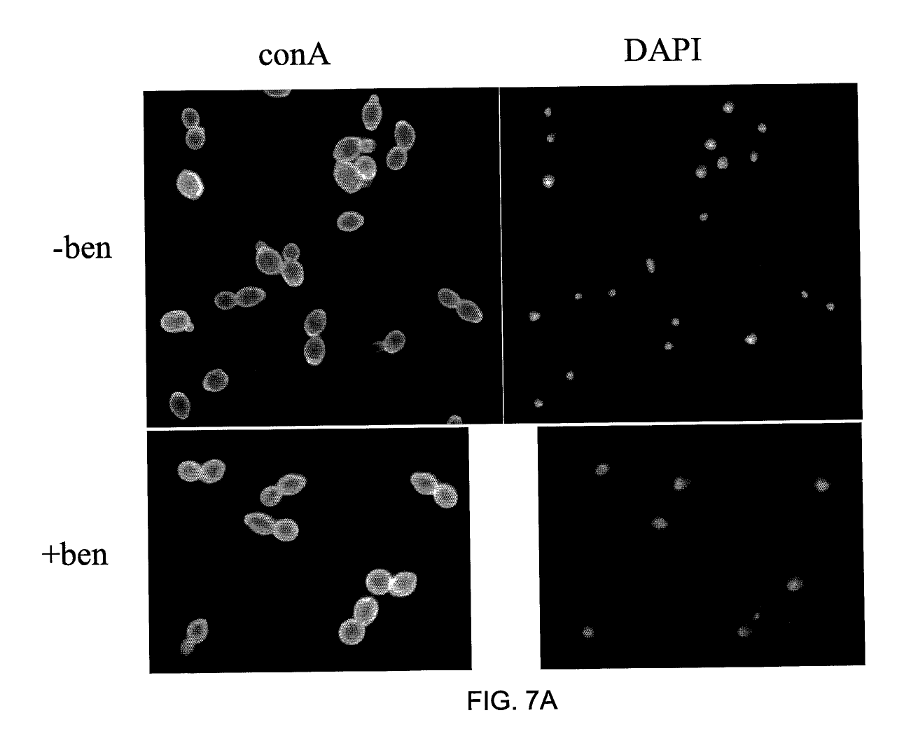

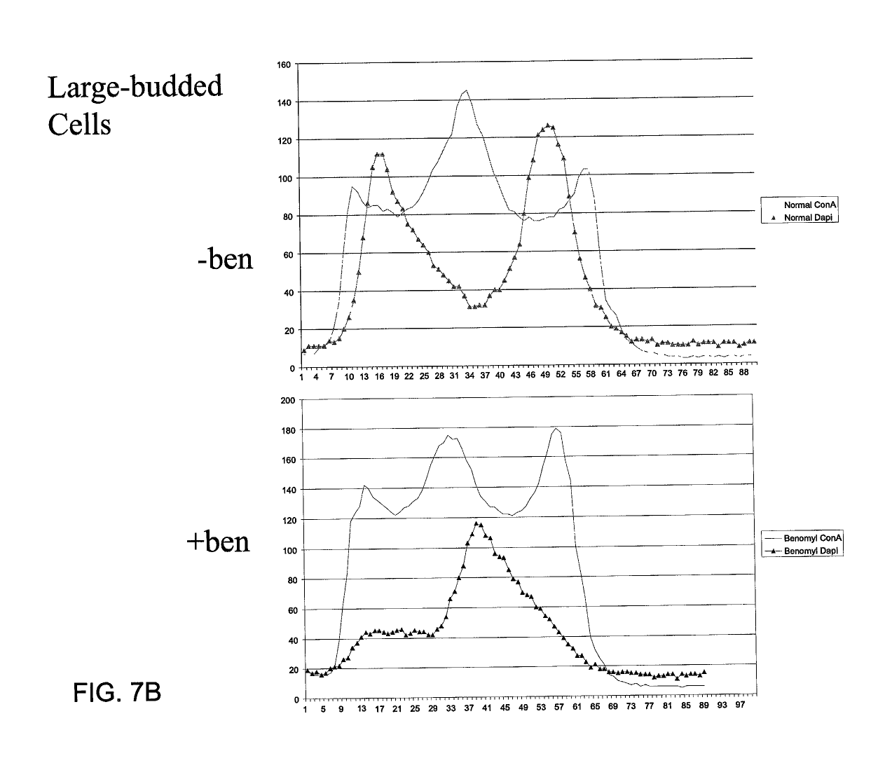

FIG. 7A shows images of two groups of cells: one which was treated with benomyl (+ben) and the other which was not treated with benomyl (−ben). As mentioned, benomyl depolymerizes microtubules and the nucleus does not divide. For each group of cells, separate images highlighting conA and DAPI were produced. As mentioned conA marks the cell wall and DAPI marks the nucleus. As can be seen, benomyl has a rather profound effect on the distribution of the nucleus and the cell wall (in the budding state). Specifically, the wildtype cells (−ben) always have two nuclei in budded cells. In benomyl treated cells, large budded cells have only one nucleus. By detecting the intensity of conA versus the intensity of DAPI, one can determine whether a given cell has one nucleus or two or more nuclei.

FIG. 7B shows a graphical representation of the cross-sectional intensity of the −ben and +ben large-budded cells. The cross-section was cut across the long axis spanning the parent and daughter cells. The vertical axis provides arbitrary fluorescence units and the horizontal axis provides distance units from an arbitrary anchor point. Importantly, in the −ben cells, one can clearly see two nuclei (DAPI peaks) located within the cell walls of the parent and daughter cells (indicated by the peaks in conA intensity). In the +ben cells, only a single DAPI peak exists —indicating that only a single nucleus exists in the budded cell. One can tell that the +ben cell is still budded because it contains three distinct conA peaks.

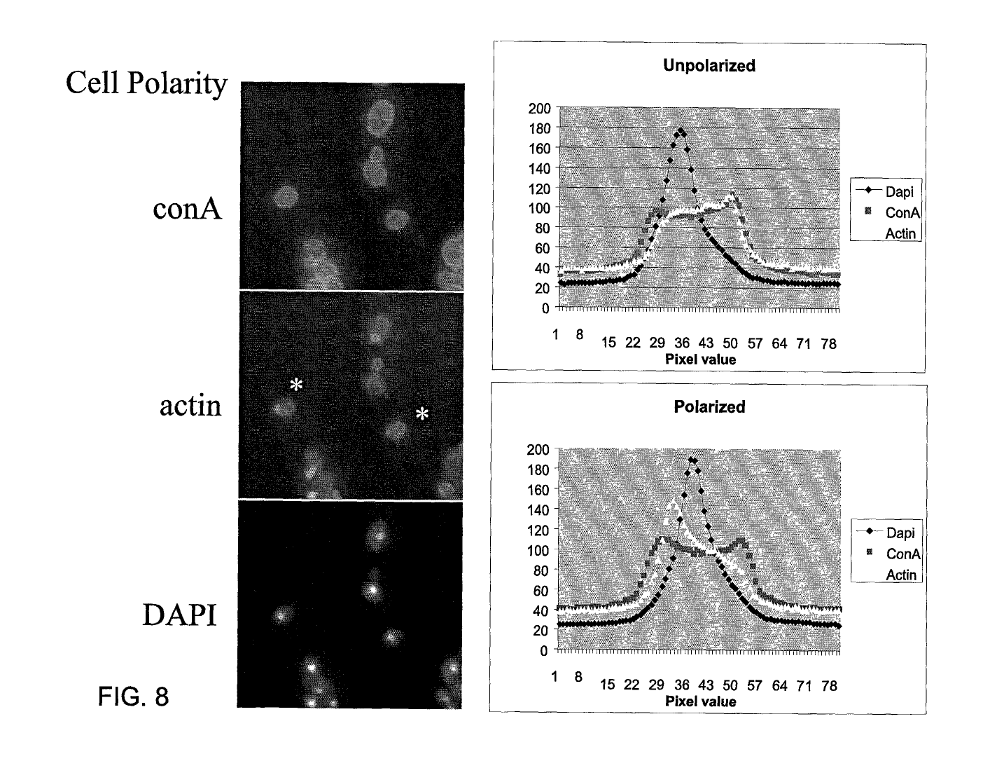

FIG. 8 illustrates the cross-sectional intensity of conA, actin, and DAPI for normal yeast cells undergoing polarization. Note that a principal characteristic of the polarized yeast cells is the location of the actin (rhodamine phalloidin) concentration with respect to the cell wall (conA) and the nucleus (DAPI).

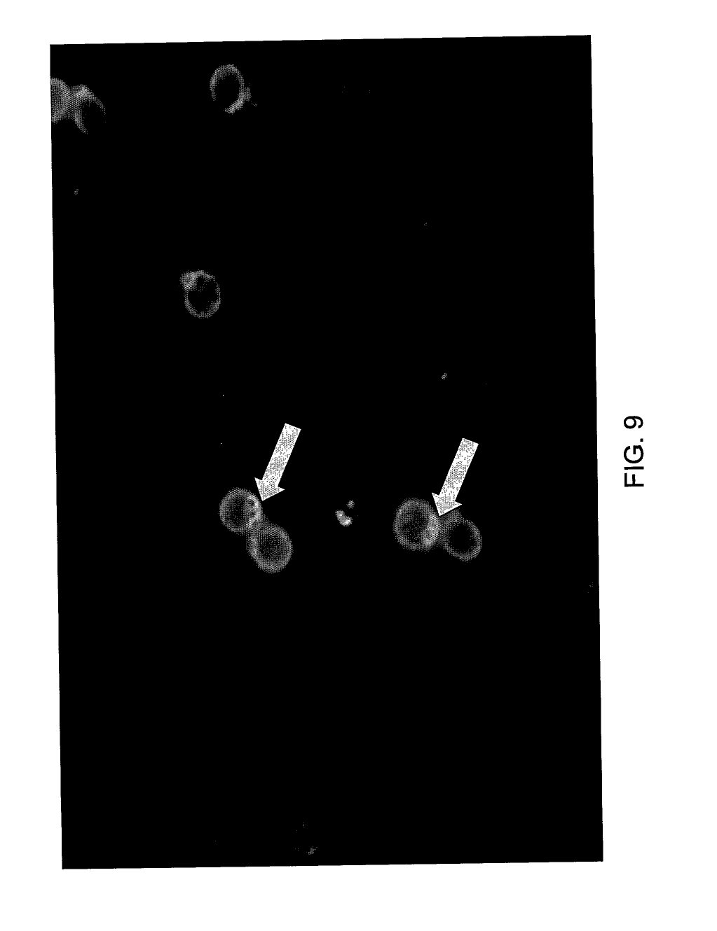

FIG. 9 shows the use of another marker, calcofluor white, to allow imaging of chitin in yeast cells. Chitin scars are generated each time a yeast cell buds. So an image of a calcofluor white marked yeast cell can show how many times the cell has budded. After about 25 divisions, a parent yeast cell will die. The positions of the bud scars are also informative. The number and position of the bud scars can tell the age of the mother cell and whether or not it is budding in a haploid (axial) or diploid (polar) manner, or any deviation from these two normal types of budding.

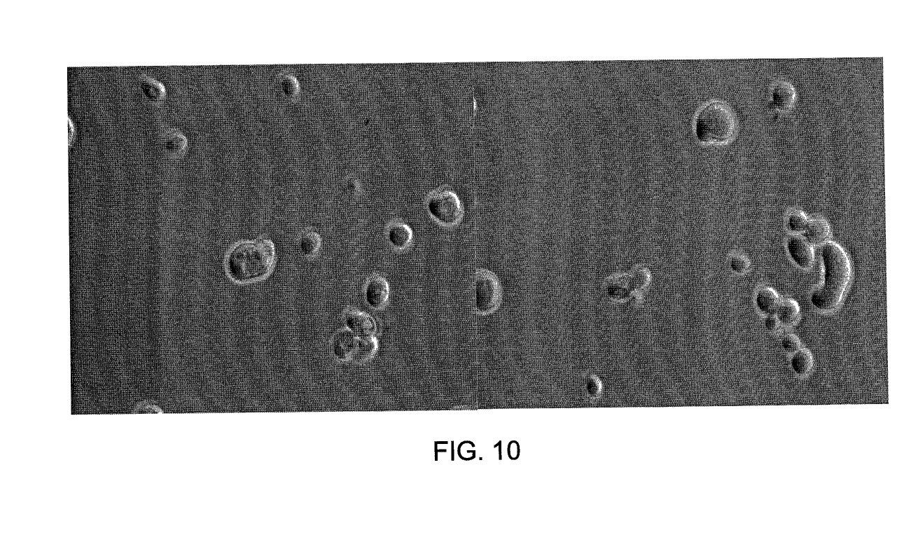

FIG. 10 shows an image of cells yeast cells exhibiting a constitutive pheromone response. Due to mutations in certain protein kinases involved in pheromone signaling, such cells have formed mating projections —even in the absence an externally present pheromone. The left and right images are two fields of the same frame. The protrusions on the cells indicate that they are in the mating phase. The image processing methods of this invention can distinguish the yeast cells exhibiting a constitutive pheromone response. MATa or MATa/MATa yeast cells exposed to alpha-factor will have a similar morphology.

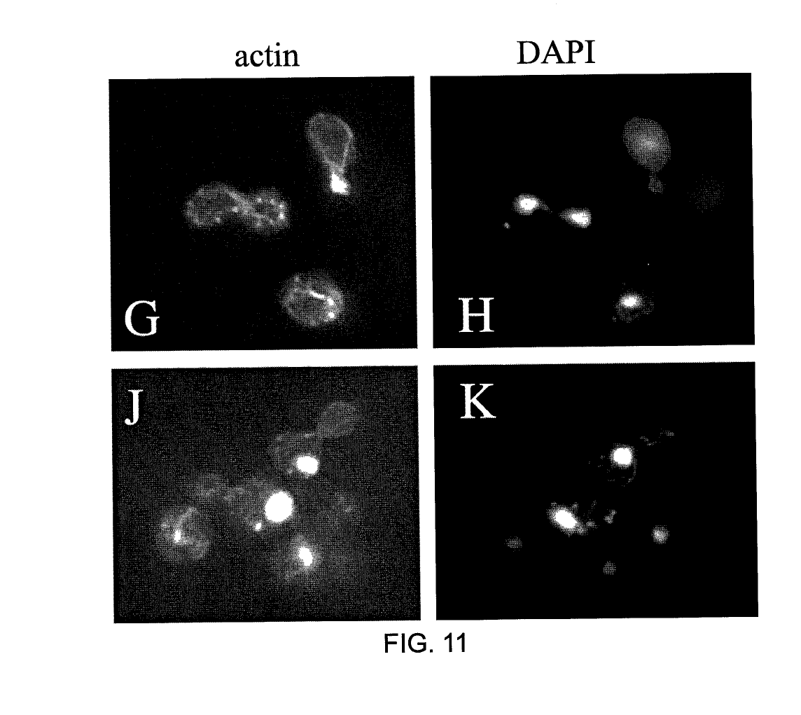

FIG. 11 shows cells having abnormal actin (actin derangement) in frame J. The large clumps of actin shown in slide J are due to protein kinase mutations. The yeast cells in the other frames are normal. Rhodamine phalloidin was used to stain the actin.

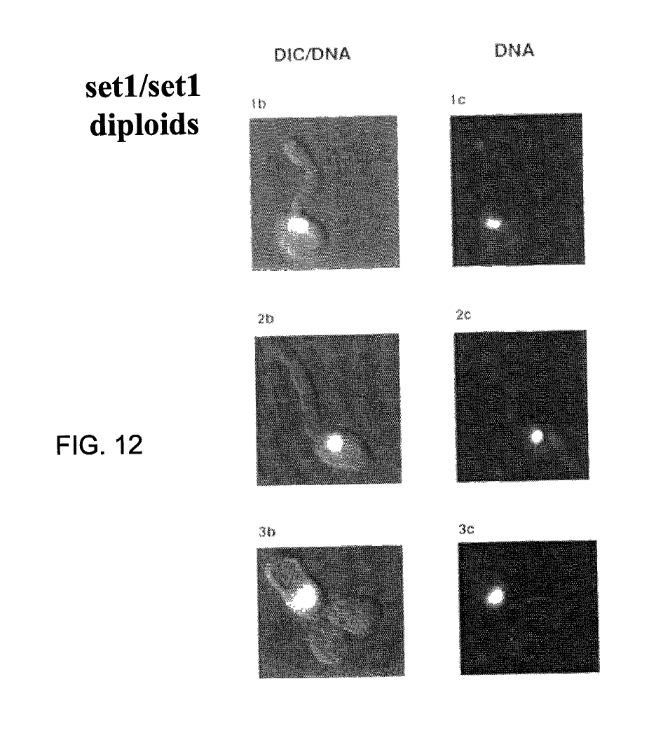

FIG. 12 shows morphological mutants in which the buds appear as long protrusions rather than the normal small oval shaped buds. In many cases, the protrusions do not contain nuclei. This mutation is caused by deletion of SET1, a transcriptional regulator that results in cell wall and mitotic defects. In this figure, DAPI was used to image the nucleus and phase microscopy was used to image the outline of the cell.

The methods of this present invention (data acquisition, image analysis, clustering, screening, etc.) may be implemented on various general or specific purpose computing systems. In one embodiment, the systems of this invention may be a specially configured personal computer or workstation. In another embodiment, the methods of this invention may be implemented on a general-purpose network host machine such as a personal computer or workstation. Further, the invention may be at least partially implemented on a card for a network device or a general-purpose computing device.

Regardless of computing device's configuration, it may employ one or more memories or memory modules configured to store program instructions for the image analysis and other functions of the present invention described herein. The program instructions may specify any one or more application programs or routines, for example. Such memory or memories may also be configured to store data structures or other specific non-program information described herein.

Because such information and program instructions may be employed to implement the systems/methods described herein, the present invention relates to machine-readable media that include program instructions, state information, etc. for performing various operations described herein. Examples of machine-readable media include, but are not limited to, magnetic media such as hard disks, floppy disks, and magnetic tape; optical media such as CD-ROM disks; magneto-optical media such as floptical disks; and hardware devices that are specially configured to store and perform program instructions, such as read-only memory devices (ROM) and random access memory (RAM). The invention may also be embodied in a carrier wave travelling over an appropriate medium such as airwaves, optical lines, electric lines, etc. Examples of program instructions include both machine code, such as produced by a compiler, and files containing higher level code that may be executed by the computer using an interpreter.

Additional information pertaining to techniques for obtaining images, analyzing those images to obtain relevant phenotypic characteristics, clustering, screening, etc. can be found in the following documents: U.S. patent application Ser. No. 09/310,879 by Vaisberg et al., and titled DATABASE METHOD FOR PREDICTIVE CELLULAR BIOINFORMATICS; U.S. patent application Ser. No. 09/311,996 by Vaisberg et al., and titled DATABASE SYSTEM INCLUDING COMPUTER FOR PREDICTIVE CELLULAR BIOINFORMATICS; and U.S. patent application Ser. No. 09/311,890 by Vaisberg et al., and titled DATABASE SYSTEM FOR PREDICTIVE CELLULAR BIOINFORMATICS. Each of these applications was filed on May 14, 1999. Each of these references is incorporated herein by reference for all purposes. Even more background information can be found in the following documents: US patent application Ser. No. 09/729,754 filed Dec. 4, 2000, naming Vaisberg et al. as inventors, and titled “CLASSIFYING CELLS BASED ON INFORMATION CONTAINED IN CELL IMAGES”; U.S. patent application Ser. No. 09/790,214 filed Feb. 20, 2001, naming Crompton et al. as inventors, and titled “METHOD AND APPARATUS FOR PREDICTIVE CELLULAR BIOINFORMATICS”; and U.S. patent application Ser. No. 09/792,012 filed Feb. 20, 2001, naming Vaisberg et al. as inventors, and titled “IMAGE ANALYSIS OF THE GOLGI COMPLEX.” Again, each of these references is incorporated herein by reference for all purposes.

Although the above has generally described the present invention according to specific systems, the present invention has a much broader range of applicability. In particular, the present invention is not limited to a particular kind of data about a particular cell, but can be applied to virtually any cellular data where an understanding about the workings of the cell is desired. Thus, in some embodiments, the techniques of the present invention could provide information about many different types or groups of cells, substances, and genetic processes of all kinds. Of course, one of ordinary skill in the art would recognize other variations, modifications, and alternatives.

What is claimed is:

1. A method of analyzing a collection of genetically modified cell strains that are congenic with a parent strain, the method comprising:
(a) receiving images of phenotypes for each of the genetically modified cell strains;
(b) analyzing the images with one or more algorithms that provide quantitative representations of the phenotypes; and
(c) comparing the quantitative representations of the phenotypes with (i) each other, (ii) a qualitative representation of the parent strain, or (iii) a quantitative representation of a phenotype of a cell that is genetically similar or identical to one or more of the cell strains.

2. The method of claim 1, wherein the genetically modified cell strains are deletion mutants having one or more genes deleted from the genome of the parent strain.

3. The method of claim 2, wherein the deletion mutants each lack a single gene present in the parent strain.

4. The method of claim 3, wherein the collection of genetically modified cell strains contains a deletion mutant for each non-essential gene in the parent strain.

5. The method of claim 4, wherein the collection of genetically modified cell strains includes the deletion mutants provided by the Saccharomyces cerevisiae Deletion Consortium.

6. The method of claim 5, wherein the collection of genetically modified cell strains further comprises mutant strains having modified, but not deleted, essential genes of Saccharomyces cerevisiae.

7. The method of claim 1, further comprising:
marking one or more cell features of the genetically modified cell strains so that said features can be highlighted in the images of the phenotypes; and
imaging the genetically modified cell strains to produce the images of the phenotypes, wherein the cell features are highlighted in the images of the phenotypes.

8. The method of claim 7, wherein the genetically modified cell strains are yeast strains and wherein marking one or more cell features comprises staining the yeast strains with a first stain for the cell wall, a second stain for the genetic material, and a third stain for the cytoskeleton.

9. The method of claim 8, wherein the first stain is concanavalin A, the second stain is DAPI, and the third stain is rhodamine phalloidin.

10. The method of claim 1, wherein analyzing the images comprises:
receiving the intensity versus position data from one or markers on the genetically modified cell strains;
quantifying geometrical information about said markers; and
quantifying biological information about said genetically modified cell strains.

11. The method of claim 10, wherein the quantitative representations of the phenotypes include one or both of the geometrical information and the biological information.

12. The method of claim 1, wherein comparing the quantitative representations of the phenotypes comprises comparing the quantitative representations of the phenotypes with each other to cluster the phenotypes and identify common functional traits shared between multiple genetic modifications.

13. The method of claim 1, wherein comparing the quantitative representations of the phenotypes comprises comparing the quantitative representations of the phenotypes with a quantitative representation of a phenotype of the cell that is genetically similar or identical to one or more of the cell strains, and wherein the cell that is genetically similar or identical has been treated with a drug or a drug candidate.

14. The method of claim 1, further comprising generating a database including records identifying the phenotypes and the quantitative representations of the phenotypes.

15. The method of claim 14, further comprising linking the database with another database containing non-morphological information about the collection of genetically modified cell strains or similar, unmodified parent strains.

16. A computer program product comprising a machine readable medium on which is provided program instructions for analyzing a collection of genetically modified cell strains that are congenic with a parent strain, the instructions comprising:
(a) code for receiving images of phenotypes for each of the genetically modified cell strains;
(b) code for analyzing the images with one or more algorithms that provide quantitative representations of the phenotypes; and
(c) code for comparing the quantitative representations of the phenotypes with (i) each other, (ii) a qualitative representation of the parent strain, or (iii) a quantitative representation of a phenotype of a cell that is genetically similar or identical to one or more of the cell strains.

17. The computer program product of claim 16, wherein the genetically modified cell strains are deletion mutants having one or more genes deleted from the genome of the parent strain.

18. The computer program product of claim 17, wherein the deletion mutants each lack a single gene present in the parent strain.

19. The computer program product of claim 18, wherein the collection of genetically modified cell strains contains a deletion mutant for each non-essential gene in the parent strain.

20. The computer program product of claim 19, wherein the collection of genetically modified cell strains includes the deletion mutants provided by the Saccharomyces cerevisiae Deletion Consortium.

21. The computer program product of claim 20, wherein the collection of genetically modified cell strains further comprises mutant strains having modified, but not deleted, essential genes of Saccharomyces cerevisiae.

22. The computer program product of claim 16, further comprising:
code for imaging the genetically modified cell strains to produce the images of the phenotypes, wherein one or more cell features are highlighted by marking in the images of the phenotypes.

23. The computer program product of claim 22, wherein the genetically modified cell strains are yeast strains and wherein marking one or more cell features was accomplished by staining the yeast strains with a first stain for the cell wall, a second stain for the genetic material, and a third stain for the cytoskeleton.

24. The computer program product of claim 23, wherein the first stain is concanavalin A, the second stain is DAPI, and the third stain is rhodamine phalloidin.

25. The computer program product of claim 16, wherein the code for analyzing the images comprises:
code for receiving the intensity versus position data from one or markers on the genetically modified cell strains;
code for quantifying geometrical information about said markers; and
code for quantifying biological information about said genetically modified cell strains.

26. The computer program product of claim 25, wherein the quantitative representations of the phenotypes include one or both of the geometrical information and the biological information.

27. The computer program product of claim 16, wherein the code for comparing the quantitative representations of the phenotypes comprises code for comparing the quantitative representations of the phenotypes with each other to cluster the phenotypes and identify common functional traits shared between multiple genetic modifications.

28. The computer program product of claim 16, wherein the code for comparing the quantitative representations of the phenotypes comprises code for comparing the quantitative representations of the phenotypes with a quantitative representation of a phenotype of the cell that is genetically similar or identical to one or more of the cell strains, and wherein the cell that is genetically similar or identical has been treated with a drug or a drug candidate.

29. The computer program product of claim 16, further code for comprising generating a database including records identifying the phenotypes and the quantitative representations of the phenotypes.

30. The computer program product of claim 29, further comprising code for linking the database with another database containing non-morphological information about the collection of genetically modified cell strains or similar, unmodified parent strains.

31. A computing device comprising a memory device configured to store at least temporarily program instructions for analyzing a collection of genetically modified cell strains that are congenic with a parent strain, the instructions comprising:
(a) code for receiving images of phenotypes for each of the genetically modified cell strains;
(b) code for analyzing the images with one or more algorithms that provide quantitative representations of the phenotypes; and
(c) code for comparing the quantitative representations of the phenotypes with (i) each other, (ii) a qualitative representation of the parent strain, or (iii) a quantitative representation of a phenotype of a cell that is genetically similar or identical to one or more of the cell strains.
